(19) United States
(12) Patent Application Publication
(10) Pub. No.: US 2025/0120899 A1
(43) Pub. Date: Apr. 17, 2025

(54) OIL-IN-WATER EMULSION COSMETIC

(71) Applicant: SHISEIDO COMPANY, LTD., Tokyo (JP)

(72) Inventors: Keita NISHIDA, Tokyo (JP); Ikuhiro SUZUKI, Tokyo (JP); Yusuke OKUYAMA, Tokyo (JP)

(73) Assignee: SHISEIDO COMPANY, LTD., Tokyo (JP)

(21) Appl. No.: 18/695,084

(22) PCT Filed: Oct. 12, 2022
(86) PCT No.: PCT/JP2022/038002

§ 371 Date: Mar. 25, 2024

(30) Foreign Application Priority Data
Oct. 25, 2021 (JP) 2021-173978

(51) Int. Cl. A61K 8/49; A61K 8/06; A61K 8/31; A61K 8/37; A61K 8/63; A61K 8/86; A61Q 19/00

(52) U.S. Cl. CPC A61K 8/4946; A61K 8/062; A61K 8/31; A61K 8/37; A61K 8/4966; A61K 8/63; A61K 8/86; A61Q 19/00

(57) ABSTRACT

[Problem] To provide an oil-in-water emulsion cosmetic having an excellent feel in use.

[Solution] An oil-in-water emulsion cosmetic including (A) a cyclic carboxamide derivative having a specific structure or its salt, (B1) a polyoxyethylene dialkyl ester or a polyoxyethylene dialkyl ether having a specific structure, (B2) a polyoxyethylene alkyl ester or a polyoxyethylene alkyl ether having a specific structure, (C) an oil, and (D) water.

### TECHNICAL FIELD

The present invention relates to an oil-in-water emulsion cosmetic.

### BACKGROUND ART

It is known that in a cosmetic, a combination of specific surfactants is blended to form an aggregate in the composition, function as an emulsifier, and impart a high moisture retaining property and good temporal stability (for example, Patent Literature 1). The users of such a cosmetic have desired a more excellent feel in use, and there has been room for further improvement in the feel in use.

Meanwhile, a cyclic carboxamide derivative is known to have an anti-wrinkle effect and a pigmentation inhibitory effect, and is proposed to be blended in a cosmetic and the like (Patent Literature 2).

### CITATION LIST

### Patent Literature

Patent Literature 1: WO 2017/131217 A
Patent Literature 2: WO 2011/040496 A

### SUMMARY OF THE INVENTION

According to the studies of the present inventors, it has been found that use of a cyclic carboxamide derivative in a cosmetic tends to cause a sticky feel and loss of smoothness of the feel. The present inventors have surprisingly found that an excellent feel in use can be achieved by using an oil-in-water cosmetic including a combination of surfactants having a specific structure and a specific cyclic carboxamide derivative. The present invention is based on these findings.

According to the present invention, the following invention is provided.
[1] An oil-in-water emulsion cosmetic including:
   (A) a cyclic carboxamide derivative or a salt of the cyclic carboxamide derivative, the cyclic carboxamide derivative represented by Formula (a):

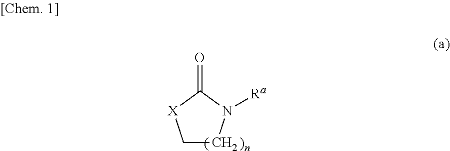

wherein
R^{a} represents a hydrocarbon group having 1 to 6 carbon atoms optionally substituted with a hydroxy group or R^{a} represents a hydrogen atom,
X represents —CH₂— or —N(R¹)— wherein R¹ represents a hydrocarbon group having 1 to 6 carbon atoms optionally substituted with a hydroxy group or R¹ represents a hydrogen atom, and
n is an integer of 1 to 3;
(B1) a polyoxyethylene dialkyl ester or a polyoxyethylene dialkyl ether represented by Formula (b1):

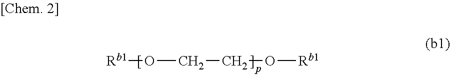

wherein
R^{b1} each independently represents a straight-chain aliphatic acid residue or a straight-chain aliphatic alcohol residue having 16 to 24 carbon atoms, and
p is an integer of 4 to 15;
(B2) a polyoxyethylene alkyl ester or a polyoxyethylene alkyl ether represented by Formula (b2):

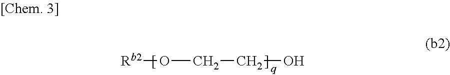

wherein
R^{b2} each independently represents a straight-chain aliphatic acid residue or a straight-chain aliphatic alcohol residue having 16 to 24 carbon atoms, and
q is an integer of 8 to 30;
(C) an oil; and
(D) water.
[2] The cosmetic according to [1],
   wherein in Formula (a) of the component (A),
   R^{a} represents a hydroxyalkyl group having 1 to 3 carbon atoms,
   X represents —CH₂— or —NH—, and
   n is 1.
[3] The cosmetic according to [1] or [2], wherein the component (A) is 1-(2-hydroxyethyl)-2-imidazolidinone.
[4] The cosmetic according to any one of [1] to [3], wherein an amount of the component (A) blended is 0.05 to 5 mass % with respect to a total amount of the cosmetic.
[5] The cosmetic according to any one of [1] to [4], further including
   (B3) a polyoxyethylene sterol ether represented by Formula (b3):

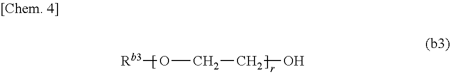

wherein
R^{b3} is a cholesterol or phytosterol residue, and
r is an integer of 5 to 30.
[6] The cosmetic according to any one of [1] to [5], wherein an amount of the component (C) blended is 50 mass % or less with respect to the total amount of the cosmetic.
[7] The cosmetic according to any one of [1] to [6], wherein the component (C) includes an ultraviolet absorber.
[8] The cosmetic according to [7], wherein an amount of the ultraviolet absorber blended is 3 to 25 mass % with respect to the total amount of the cosmetic.

According to the present invention, an oil-in-water emulsion cosmetic having an excellent feel in use can be provided. In particular, the feel during application is smooth, and a sticky feel after application is suppressed.

### BEST MODE FOR CARRYING OUT THE INVENTION

The present invention relates to an oil-in-water emulsion cosmetic (hereinafter, sometimes referred to as a cosmetic) including (A) a cyclic carboxamide derivative having a specific structure or its salt, (B1) a polyoxyethylene dialkyl ester or a polyoxyethylene dialkyl ether having a specific structure, (B2) a polyoxyethylene alkyl ester or a polyoxyethylene alkyl ether having a specific structure, (C) an oil, and (D) water.

### (A) Cyclic Carboxamide Derivative or Its Salt

The cosmetic according to the present invention includes a cyclic carboxamide derivative represented by Formula (a) or its salt (hereinafter, sometimes referred to as a component (A), and the same applies to other components).

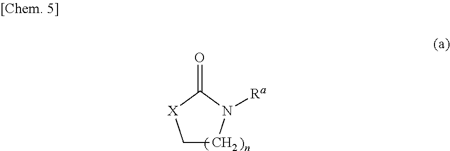

In Formula (a),
R^{a} represents a hydrocarbon group having 1 to 6 carbon atoms optionally substituted with a hydroxy group or R^{a} represents a hydrogen atom,
X represents —CH₂— or —N(R¹)— wherein R¹ represents a hydrocarbon group having 1 to 6 carbon atoms optionally substituted with a hydroxy group or R¹ represents a hydrogen atom, and
n is an integer of 1 to 3

The hydrocarbon group is not particularly limited, and may be, for example, an alkyl group, a cycloalkyl group, an alkenyl group, an alkynyl group, a cycloalkylalkyl group, a haloalkyl group, an alkoxyalkyl group, or an alkoxycarbonylalkyl group, and is preferably an alkyl group.

In a preferred embodiment, in Formula (a) of the component (A),
R^{a} represents a hydroxyalkyl group having 1 to 3 carbon atoms,
X represents —CH₂— or —NH—, and
n is 1.

Specific examples of the cyclic carboxamide derivative represented by Formula (a) Include the following.

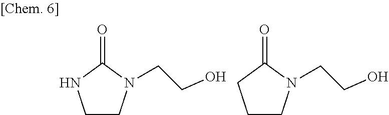

The component (A) is most preferably 1-(2-hydroxyethyl)-2-imidazolidinone.

The component (A) may be a salt of the cyclic carboxamide derivative represented by Formula (a). The kind of the salt is not particularly limited as long as the salt is pharmacologically acceptable, and the salt may be an inorganic salt or an organic salt. Examples of the inorganic salt include a hydrochloride, a sulfate, a phosphate, a hydrobromide, a sodium salt, a potassium salt, a magnesium salt, a calcium salt, a magnesium salt, and an ammonium salt. Examples of the organic salt include an acetate, a lactate, a maleate, a fumarate, a tartrate, a methanesulfonate, a p-toluenesulfonate, a triethanolamine salt, and an amino acid salt.

One or more kinds of the component (A) can be blended. The amount of the component (A) blended is preferably 0.05 to 5 mass %, more preferably 0.08 to 3.5 mass %, and still more preferably 0.5 to 3.0 mass % with respect to the total amount of the cosmetic. Within this range, a further improvement can be achieved against deterioration of smoothness during application and a sticky feel after application that are caused by the component (A).

### (B1) Polyoxyethylene Dialkyl Ester or Polyoxyethylene Dialkyl Ether

The cosmetic according to the present invention includes a polyoxyethylene dialkyl ester or a polyoxyethylene dialkyl ether represented by Formula (b1).

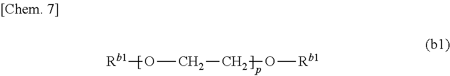

In Formula (b1),
R^{b1} each independently represents a straight-chain aliphatic acid residue or a straight-chain aliphatic alcohol residue having 16 to 24 carbon atoms, and
p is an integer of 4 to 15.

In Formula (b1), the polyoxyethylene chain and the two alkyl groups may be bonded in the mode of an ester, ether, or a combination of an ester and an ether. The two R^{b1} may be the same or different.

Examples of the component (B1) include polyoxyethylene (4 mol) distearic acid (for example, Emalex 200DIS by Nihon Emulsion Co., Ltd.), polyoxyethylene (6 mol) distearic acid (for example, Emalex 300DIS by Nihon Emulsion Co., Ltd.), polyoxyethylene (8 mol) distearic acid (for example, Emalex 400DIS by Nihon Emulsion Co., Ltd.), polyoxyethylene (12 mol) distearic acid (for example, Emalex 600DIS by Nihon Emulsion Co., Ltd.), steareth-4 stearate (for example, Emalex SWS-4 by Nihon Emulsion Co., Ltd.), steareth-6 stearate (for example, Emalex SWS-6 by Nihon Emulsion Co., Ltd.), steareth-9 stearate (for example, Emalex SWS-9 by Nihon Emulsion Co., Ltd.), and polyoxyethylene (8 mol) dibehenyl ether.

One or more kinds of the component (B1) can be blended. The amount of the component (B1) blended is preferably 0.05 to 3 mass %, and more preferably 0.1 to 1 mass % with respect to the total amount of the cosmetic. Within this range, a further improvement can be achieved against deterioration of smoothness during application and a sticky feel after application that are caused by the component (A), and furthermore, the temporal stability of the cosmetic can be improved.

### (B2) Polyoxyethylene Alkyl Ester or Polyoxyethylene Alkyl Ether

The cosmetic according to the present invention includes a polyoxyethylene alkyl ester or a polyoxyethylene alkyl ether represented by Formula (b2).

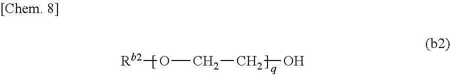

In Formula (b2),
R^{b2} each independently represents a straight-chain aliphatic acid residue or a straight-chain aliphatic alcohol residue having 16 to 24 carbon atoms, and
q is an integer of 8 to 30.

Examples of the component (B2) include polyoxyethylene (5 mol) behenyl ether (for example, Nikkol BB-5 by Nikko Chemicals Co., Ltd.), polyoxyethylene (10 mol) behenyl ether (for example, Nikkol BB-10 by Nikko Chemicals Co., Ltd.), polyoxyethylene (20 mol) behenyl ether (for example, Nikkol BB-20 by Nikko Chemicals Co., Ltd.), polyoxyethylene (10 mol) stearyl ether (for example, Emalex 610 by Nihon Emulsion Co., Ltd.), and polyoxyethylene (7 mol) cetyl ether (for example, Emalex 107 by Nihon Emulsion Co., Ltd.).

One or more kinds of the component (B2) can be blended. The amount of the component (B2) blended is preferably 0.05 to 3 mass %, and more preferably 0.1 to 1 mass % with respect to the total amount of the cosmetic. Within this range, a further improvement can be achieved against deterioration of smoothness during application and a sticky feel after application that are caused by the component (A), and furthermore, the temporal stability of the cosmetic can be improved.

### (B3) Polyoxyethylene Sterol Ether

The cosmetic according to the present invention can further include a polyoxyethylene sterol ether represented by Formula (b3).

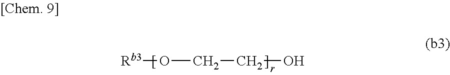

In Formula (b3),
R^{b3} is a cholesterol or phytosterol residue, and
r is an integer of 5 to 30.

Examples of the component (B3) include polyoxyethylene (5 mol) phytosterol (for example, Nikkol BPS-5 by Nikko Chemicals Co., Ltd.), polyoxyethylene (10 mol) phytosterol (for example, Nikkol BPS-10 by Nikko Chemicals Co., Ltd.), polyoxyethylene (20 mol) phytosterol (for example, Nikkol BPS-20 by Nikko Chemicals Co., Ltd.), polyoxyethylene (30 mol) phytosterol (for example, Nikkol BPS-30 by Nikko Chemicals Co., Ltd.), and polyoxyethylene (10 mol) cholesterol (for example, Emalex CS-10 by Nihon Emulsion Co., Ltd.).

One or more kinds of the component (B3) can be blended. The amount of the component (B3) blended is preferably 0.05 to 5 mass %, and more preferably 0.1 to 3 mass % with respect to the total amount of the cosmetic.

The components (B1) and (B2) (and the component (B3) as necessary) can form an aggregate in the composition to function as an emulsifier. By using such an emulsifier, the moisture retaining property of the skin can be enhanced, and the temporal stability of the cosmetic can be improved.

The total amount of the components (B1), (B2), and (B3) blended is preferably 0.1 to 10 mass %, and more preferably 0.5 to 3 mass % with respect to the total amount of the cosmetic.

### (C) Oil

The cosmetic according to the present invention includes (C) an oil. Examples of the component (C) include hydrocarbon oils, ester oils, silicone oils, higher fatty acids, higher alcohols, liquid oils, solid oils, semi-solid oils, and ultraviolet absorbers, and hydrocarbon oils and ester oils are preferable.

Examples of the hydrocarbon oils include isododecane, isohexadecane, isoparaffin, mineral oil (liquid paraffin), ozokerite, squalane, pristane, paraffin, ceresin, squalene, petrolatum, microcrystalline wax, and hydrogenated polydecene.

Examples of the ester oils include octyl octanoate, nonyl nonanoate, cetyl octanoate, isopropyl myristate, octyldodecyl myristate, isopropyl palmitate, ethylhexyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, tripropylene glycol pivalate, diisostearyl malate, glycerin di-2-heptylundecanoate, glycerin diisostearate, trimethylolpropane tri-2-ethylhexanoate, trimethylolpropane triisostearate, pentaerythritol tetra-2-ethylhexanoate, glyceryl tri-2-ethylhexanoate (triethylhexanoin), glyceryl trioctanoate, glyceryl triisopalmitate, trimethylolpropane triisostearate, cetyl 2-ethylhexanoate-2-ethylhexyl palmitate, glyceryl trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamate-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, cetyl ethylhexanoate, and phytosteryl macadamiate.

Examples of the silicone oils include chain polysiloxanes (such as dimethicone, diphenylsiloxy phenyl trimethicone, and diphenylpolysiloxane), cyclic polysiloxanes (such as octamethylcyclotetrasiloxane, decamethylcyclopentasiloxane, and dodecamethylcyclohexasiloxane), silicone resins forming a three-dimensional network structure, silicone rubber, various modified polysiloxanes (such as an amino-modified polysiloxane, a polyether-modified polysiloxane, an alkyl-modified polysiloxane, and a fluorine-modified polysiloxane), and acrylic silicones, and chain polysiloxanes are preferable.

Examples of the higher fatty acids include lauric acid, myristic acid, palmitic acid, stearic acid, behenic acid, oleic acid, undecylenic acid, tall oil acid, isostearic acid, linolic acid, linoleic acid, eicosapentaenoic acid (EPA), and docosahexaenoic acid (DHA).

Examples of the higher alcohols include straight-chain alcohols (such as lauryl alcohol, cetyl alcohol, stearyl alcohol, behenyl alcohol, myristyl alcohol, oleyl alcohol, and cetostearyl alcohol) and branched alcohols (such as lanolin alcohol, cholesterol, phytosterol, hexyldodecanol, isostearyl alcohol, and octyldodecanol).

Examples of the liquid oils include avocado oil, camellia oil, macadamia nut oil, corn oil, olive oil, rapeseed oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, paulownia oil, Japanese tung oil, jojoba oil, germ oil, and triglycerin oil. Examples of the solid oils include cacao butter, coconut oil, hardened coconut oil, palm oil, palm kernel oil, hydrogenated palm oil, Japan wax kernel oil, hardened oils, Japan wax, and hardened castor oil. Examples of the semi-solid oils include shea butter, partially hydrogenated coconut oil, and partially hydrogenated jojoba oil.

Examples of the ultraviolet absorbers include benzoic acid derivatives, salicylic acid derivatives, cinnamic acid derivatives, dibenzoylmethane derivatives, β,β-diphenylacrylate derivatives, benzophenone derivatives, benzylidene camphor derivatives, phenylbenzimidazole derivatives, triazine derivatives, phenylbenzotriazole derivatives, anthranil derivatives, imidazoline derivatives, benzalmalonate derivatives, and 4,4-diarylbutadiene derivatives.

Examples of the benzoic acid derivatives include ethyl para-aminobenzoate (PABA), ethyl-dihydroxypropyl PABA, ethylhexyl-dimethyl PABA, glyceryl PABA, PEG-25-PABA, and diethylamino hydroxybenzoyl hexyl benzoate. Examples of the salicylic acid derivatives include homosalate, ethylhexyl salicylate, dipropylene glycol salicylate, and TEA salicylate.

Examples of the cinnamic acid derivatives include octyl methoxycinnamate or ethylhexyl methoxycinnamate, isopropyl methoxycinnamate, isoamyl methoxycinnamate, cinnoxate, DEA methoxycinnamate, diisopropyl methyl cinnamate, glyceryl-ethylhexanoate-dimethoxycinnamate, and di-(2-ethylhexyl)-4′-methoxybenzalmalonate. Examples of the benzoylmethane derivatives include 4-tert-butyl-4′-methoxydibenzoylmethane. Examples of the 0,0-diphenylacrylate derivatives include octocrylene. Examples of the benzophenone derivatives include benzophenone-1, benzophenone-, benzophenone-3 or oxybenzone, benzophenone-4, benzophenone-5, benzophenone-6, benzophenone-8, benzophenone-9, and benzophenone-12. Examples of the benzylidene camphor derivatives include 3-benzylidene camphor, 4-methylbenzylidene camphor, benzylidene camphor sulfonic acid, camphor benzalkonium methosulfate, terephthalylidene dicamphor sulfonic acid, and polyacrylamidomethyl benzylidene camphor. Examples of the phenylbenzimidazole derivatives include phenylbenzimidazole sulfonic acid and disodium phenyl dibenzimidazole tetrasulfonate. Examples of the triazine derivatives include anisotriazine, ethylhexyl triazone, diethylhexyl butamidotriazone, and 2,4,6-tris(diisobutyl-4′-aminobenzalmalonate)-s-triazine. Examples of the phenylbenzotriazole derivatives include drometrizole trisiloxane and methylenebis. Examples of the anthranil derivatives include menthyl anthranilate. Examples of the imidazoline derivatives include ethylhexyl dimethoxybenzylidene dioxoimidazoline propionate. Examples of the benzalmalonate derivatives include polyorganosiloxanes having a benzalmalonate functional group. Examples of the 4,4-diarylbutadiene derivatives include 1,1-dicarboxy(2,2′-dimethylpropyl)-4,4-diphenylbutadiene.

One or more kinds of the component (C) can be blended. The amount of the component (C) blended is preferably 50 mass % or less, more preferably 1 to 30 mass %, and still more preferably 1 to 15 mass % with respect to the total amount of the cosmetic.

It is generally known that if an ultraviolet absorber as an oil is blended in a cosmetic, the feel in use tends to deteriorate to cause, for example, a sticky feel. According to the present invention, a cosmetic can be provided that can realize a sufficient feel in use even if an ultraviolet absorber is included. Therefore, in a preferred embodiment of the present invention, the component (C) includes an ultraviolet absorber. In this case, the amount of the ultraviolet absorber blended is preferably 3 to 25 mass %, and more preferably 5 to 20 mass % with respect to the total amount of the cosmetic.

### (D) Water

The cosmetic according to the present invention includes (D) water. As the water, water used in cosmetics, quasi-drugs, and the like can be used, and for example, purified water, ion-exchanged water, tap water, or the like can be used.

The amount of the water blended is preferably 20 to 95 mass %, and more preferably 60 to 90 mass % with respect to the total amount of the cosmetic according to the present invention.

In the cosmetic in the present invention, an optional component usually used in cosmetics and pharmaceuticals can be blended in addition to the above-described components. Examples of the optional component include the following components, and one or more kinds of them can be blended as long as an effect of the present invention is exhibited.

Examples of the moisturizer include polyethylene glycol, propylene glycol, dipropylene glycol (DPG), glycerin, 1,3-butylene glycol (BG), erythritol, xylitol, sorbitol, maltitol, chondroitin sulfate, hyaluronic acid, mucoitinsulfuric acid, charonin acid, atelocollagen, cholesteryl-12-hydroxystearate, sodium lactate, bile acid salts, dl-pyrrolidone carboxylates, short-chain soluble collagen, a chestnut rose extract, a yarrow extract, and a melilot extract.

Examples of the lower alcohol include ethanol, 1-propanol, 2-propanol, isobutyl alcohol, and t-butyl alcohol.

Examples of the thickener include gum arabic, carrageenan, karaya gum, tragacanth gum, carob gum, quince seed (Pyrus cydonia), casein, dextrin, gelatin, sodium pectinate, sodium alginate, methylcellulose, ethylcellulose, CMC, hydroxyethylcellulose, hydroxypropylcellulose, PVA, PVM, PVP, sodium polyacrylate, a carboxyvinyl polymer, locust bean gum, guar gum, tamarind gum, dialkyldimethylammonium sulfate cellulose, xanthan gum, magnesium aluminum silicate, bentonite, hectorite, magnesium aluminum silicate (VEEGUM), laponite, and silicic anhydride.

Examples of the sequestering agent include 1-hydroxyethane-1,1-diphosphonic acid, 1-hydroxyethane-1,1-diphosphonic acid tetrasodium salt, edetate disodium, edetate trisodium, edetate tetrasodium, sodium citrate, sodium polyphosphate, sodium metaphosphate, gluconic acid, phosphoric acid, citric acid, succinic acid, edetic acid, and trisodium hydroxyethyl ethylenediamine triacetate.

Examples of the neutralizer include 2-amino-2-methyl-1-propanol, 2-amino-2-methyl-1,3-propanediol, potassium hydroxide, sodium hydroxide, triethanolamine, and sodium carbonate.

Examples of a pH adjuster include buffers such as lactic acid-sodium lactate, citric acid-sodium citrate, and succinic acid-sodium succinate.

Examples of the antioxidant include dibutylhydroxytoluene, butylhydroxyanisole, sodium pyrosulfite, and gallic acid esters.

Examples of the preservative include paraoxybenzoic acid esters such as methylparaben, ethylparaben, and butylparaben, benzoic acid, salicylic acid, sorbic acid, parachlorometacresol, hexachlorophene, benzalkonium chloride, chlorhexidine chloride, trichlorocarbanilide, photosensitizers, and phenoxyethanol.

Examples of the drug include ascorbic acid (vitamin C), tranexamic acid, kojic acid, ellagic acid, arbutin, alkoxysalicylic acid, glycyrrhizic acid, tocopherol, retinol, salts and derivatives thereof (for example, sodium L-ascorbate, L-ascorbic acid ester magnesium salt, L-ascorbic acid glucoside, 2-O-ethyl-L-ascorbic acid, 3-O-ethyl-L-ascorbic acid, 4-methoxysalicylic acid sodium salt, 4-methoxysalicylic acid potassium salt, dipotassium glycyrrhizinate, stearyl glycyrrhizinate, tocopherol acetate, retinol acetate, and retinol palmitate), caffeine, tannin, verapamil and its derivatives, a licorice extract, glabridin, a hot water extract of Pyracantha fortuneana seed, various herbal medicines, hydrolyzed silk, hydrolyzed conchiolin, a tea extract, a Potentilla erecta root extract, an Angelica keiskei leaf/stem extract, an Aloe barbadensis leaf extract, a cherry leaf extract, an Angelica acutiloba Kitagawa root extract, a Citrus depressa peel extract, an Iris florentina root extract, an Eucheuma serra/Grateloupia sparsa/Saccharina angustata/Ulva linza/Undaria pinnatifida extract, a Typha angustifolia spike extract, an Isodonis japonicus leaf/stalk extract, a Camellia japonica seed extract, a Saccharina angustata/Undaria pinnatifida extract, a Bupleurum falcatum root extract, a Nasturtium officinale leaf/stem extract, a Cinnamomum cassia bark extract, a rosemary leaf oil, a lavender oil, glutamic acid, trimethylglycine, and menthoxypropanediol.

In addition, a powder component, an aroma chemical, and the like can also be appropriately blended.

Examples of the cosmetic according to the present invention include skin care cosmetics (such as lotions, milky lotions, creams, beauty essences, packs, and masks), makeup cosmetics (such as foundations and makeup bases), skin cleaning agents (such as face washes and makeup removers), sunscreen cosmetics, and ointments. These embodiments are merely examples, and the cosmetic according to the present invention is not limited to these embodiments.

The method of emulsification for the cosmetic of the present invention is not particularly limited, and the cosmetic can be produced in accordance with a conventional method.

The average emulsified particle size is not particularly limited, but is preferably 0.1 to 30 μm, and more preferably 0.5 to 20 μm or less. The average emulsified particle size can be measured using a microscope.

The viscosity is not particularly limited, but is preferably 100 to 100,000 mPa·s, more preferably 500 to 30,000 mPa·s, and particularly preferably 1,000 to 15,000 mPa·s. The viscosity can be measured at 30° C. using a B type viscometer.

### EXAMPLES

The present invention will be described specifically with reference to the following Examples, but the present invention is not limited to these Examples. The content is shown in mass % with respect to the total amount unless otherwise specified.

### Examples 1 to 4 and Comparative Examples 1 to 5

Cosmetics of Examples 1 to 4 and Comparative Examples 1 to 5 were prepared at combinations shown in Table 1. The numerical values in the table are shown in mass %.

TABLE 1

| | | | | | | Compar- | Compar- | Compar- | Compar- | | Compar- |
|---|---|---|---|---|---|---|---|---|---|---|---|
| | | | Exam- | Exam- | Exam- | ative | ative | ative | ative | Exam- | ative |
| | | | ple 1 | ple 2 | ple 3 | Example 1 | Example 2 | Example 3 | Example 4 | ple 4 | Example 5 |
| Composition | (A) | 1-(2-Hydroxyethyl)-2- | 3 | 2 | 1 | 3 | 3 | 2 | 1 | 3 | 3 |
| | | imidazolidinone | | | | | | | | | |
| | (B1) | PEG-6 distearate | 0.3 | 0.3 | 0.3 | — | 0.3 | 0.3 | 0.3 | 0.3 | 0.3 |
| | (B2) | beheneth-10 | 0.3 | 0.3 | 0.3 | 0.3 | — | — | — | 0.3 | — |
| | (B3) | PEG-10 phytosterol | 0.8 | 0.8 | 0.8 | 0.8 | 0.8 | 0.8 | 0.8 | 0.8 | 0.8 |
| | (C) | Triethylhexanoin | 4 | 4 | 4 | 4 | 4 | 4 | 4 | — | — |
| | (C) | Hydrogenated polydecene | 4 | 4 | 4 | 4 | 4 | 4 | 4 | — | — |
| | (C) | Ethylhexyl methoxycinnamate | — | — | — | — | — | — | — | 7.5 | 7.5 |
| | (C) | Bis-ethylhexyloxyphenol | — | — | — | — | — | — | — | 1 | 1 |
| | | methoxyphenyl triazine | | | | | | | | | |
| | (D) | Water | Balance | Balance | Balance | Balance | Balance | Balance | Balance | Balance | Balance |

| | | | | | | | | | | |
|---|---|---|---|---|---|---|---|---|---|---|
| | Dipropylene glycol | 5 | 5 | 5 | 5 | 5 | 5 | 5 | 5 | 5 |
| | Total | 100 | 100 | 100 | 100 | 100 | 100 | 100 | 100 | 100 |
| Evaluation | Absence of sticky feel | A | A | A | C | C | C | C | B | D |
| | Smoothness of feel | A | A | A | C | C | C | C | A | C |

The cosmetic prepared above was applied to the skin of 20 expert panelists, and the panelists evaluated the “absence of a sticky feel” after the application and the “smoothness of the feel” during the application. Evaluation was determined in accordance with the following criteria on the basis of the evaluation of each expert panelist. The obtained results are as shown in Table 1.

### [Absence of Sticky Feel]

A: Fifteen or more of the 20 panelists answered that a sticky feel was absent.
B: Twelve or more and 14 or less of the 20 panelists answered that a sticky feel was absent.
C: Seven or more and 11 or less of the 20 panelists answered that a sticky feel was absent.
D: Six or less of the 20 panelists answered that a sticky feel was absent.

### [Smoothness of Feel]

A: Fifteen or more of the 20 panelists answered that smoothness of the feel was present.
B: Twelve or more and 14 or less of the 20 panelists answered that smoothness of the feel was present.
C: Seven or more and 11 or less of the 20 panelists answered that smoothness of the feel was present.
D: Six or less of the 20 panelists answered that smoothness of the feel was present.

1. An oil-in-water emulsion cosmetic comprising:
(A) a cyclic carboxamide derivative or a salt of the cyclic carboxamide derivative, the cyclic carboxamide derivative represented by Formula (a): 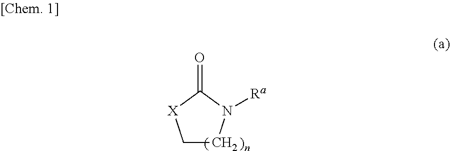
wherein
R^{a} represents a hydrocarbon group having 1 to 6 carbon atoms optionally substituted with a hydroxy group or R^{a} represents a hydrogen atom,
X represents —CH₂— or —N(R¹)— wherein R¹ represents a hydrocarbon group having 1 to 6 carbon atoms optionally substituted with a hydroxy group or R¹ represents a hydrogen atom, and
n is an integer of 1 to 3;
(B1) a polyoxyethylene dialkyl ester or a polyoxyethylene dialkyl ether represented by Formula (b1): 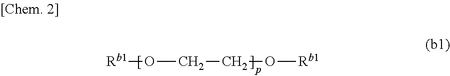
wherein
R^{b1} each independently represents a straight-chain aliphatic acid residue or a straight-chain aliphatic alcohol residue having 16 to 24 carbon atoms, and
p is an integer of 4 to 15;
(B2) a polyoxyethylene alkyl ester or a polyoxyethylene alkyl ether represented by Formula (b2): 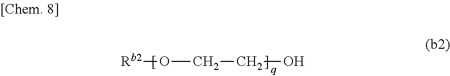
wherein
R^{b2} each independently represents a straight-chain aliphatic acid residue or a straight-chain aliphatic alcohol residue having 16 to 24 carbon atoms, and
q is an integer of 8 to 30;
(C) an oil; and
(D) water.

2. The cosmetic according to claim 1,
wherein in Formula (a) of the component (A),
R^{a} represents a hydroxyalkyl group having 1 to 3 carbon atoms,
X represents —CH₂— or —NH—, and
n is 1.

3. The cosmetic according to claim 1, wherein the component (A) is 1-(2-hydroxyethyl)-2-imidazolidinone.

4. The cosmetic according to claim 1, wherein an amount of the component (A) blended is 0.05 to 5 mass % with respect to a total amount of the cosmetic.

5. The cosmetic according to claim 1, further comprising
(B3) a polyoxyethylene sterol ether represented by Formula (b3): 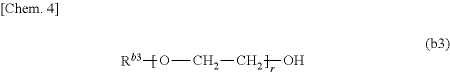
wherein
R^{b3} is a cholesterol or phytosterol residue, and
r is an integer of 5 to 30.

6. The cosmetic according to claim 1, wherein an amount of the component (C) blended is 50 mass % or less with respect to the total amount of the cosmetic.

7. The cosmetic according to claim 1, wherein the component (C) includes an ultraviolet absorber.

8. The cosmetic according to claim 7, wherein an amount of the ultraviolet absorber blended is 3 to 25 mass % with respect to the total amount of the cosmetic.
